(19) United States
(12) Patent Application Publication
(10) Pub. No.: US 2026/0328654 A1
(43) Pub. Date: Oct. 22, 2026

(54) LIGHTWEIGHT AND COMPACT LUNAR REGOLITH SAMPLING SYSTEMS MOUNTABLE ON ROVERS

(71) Applicant: SOUTHEAST UNIVERSITY, Nanjing (CN)

(72) Inventors: Aiguo SONG, Nanjing (CN); Ziyi ZHAO, Nanjing (CN); Yifei XING, Nanjing (CN); Lifeng ZHU, Nanjing (CN)

(73) Assignee: SOUTHEAST UNIVERSITY, Nanjing (CN)

(21) Appl. No.: 19/696,274

(22) Filed: Jun. 2, 2026

Related U.S. Application Data
(63) Continuation of application No. PCT/CN2024/088235, filed on Apr. 17, 2024.

(30) Foreign Application Priority Data
Apr. 2, 2024 (CN) 202410391874.8

(51) Int. Cl. G01N 1/08; B25J 9/10; B25J 9/12; B25J 9/16; G01N 33/24

(52) U.S. Cl. CPC G01N 1/08; B25J 9/102; B25J 9/126; B25J 9/1633; B25J 9/1679; G01N 33/24

(57) ABSTRACT

A lightweight and compact lunar regolith sampling system mountable on a rover is provided, comprising a three-degree-of-freedom manipulator based on open cylindrical thin-walled rods and a control unit based on three-dimensional force/torque information; wherein the three-degree-of-freedom manipulator based on the open cylindrical thin-walled rods includes a translation mechanism, a side rotation mechanism, and a rear rotation mechanism, and a sensor and a sampler are connected below the three-degree-of-freedom manipulator; and the control unit based on the three-dimensional force/torque information collects force feedback information, sends the force feedback information to a mini computer NUC, and the mini computer NUC reads an output shaft position of a servo motor by using CAN bus communication, generates a real-time control instruction in combination with a compliance control algorithm, and sends the real-time control instruction to the servo motor by using the CAN bus communication to realize a control closed loop. The present disclosure has lightweight characteristics and strong storage capacities on the basis of ensuring structural strength, monitors an operating environment and adjusts a sampling strategy in real time by using three-dimensional force/torque information, effectively copes with a complex operating environment, and significantly improves safety and flexibility of a sampling operation.

### CROSS-REFERENCE TO RELATED APPLICATIONS

This application is a continuation application of international patent application No. PCT/CN2024/088235 filed on Apr. 17, 2024, which claims priority to Chinese Patent Application No. 202410391874.8 filed on Apr. 2, 2024, the entire contents of which are hereby incorporated by reference.

### TECHNICAL FIELD

The present disclosure relates to the technical field of deep space exploration equipment, and in particular to a lightweight and compact lunar regolith sampling system mountable on a rover.

### BACKGROUND

Lunar exploration has become a key mission for exploring major scientific issues such as the formation and evolution of the universe and the origin of life. Mobile sampling by a rover has become an important development trend. However, a rover is limited by power supply and payload capacity, and a lunar surface environment is unknown and complex, requiring a sampling system to be lightweight and compact and have an environmental perception capability. Existing sampling systems mostly adopt a rigid manipulator, visual monitoring, and open-loop control, suffering from problems such as being heavy in weight, being incapable of sensing a working environment beneath lunar soil, and being incapable of adjusting a sampling trajectory in real time.

Therefore, a lightweight and compact lunar regolith sampling system mountable on a rover needs to be developed.

### SUMMARY

To solve the above problems, the present disclosure discloses a lightweight and compact lunar regolith sampling system mountable on a rover. The system has lightweight characteristics and strong storage capacity while ensuring structural strength. It utilizes three-dimensional force/torque information to monitor an operating environment and adjust a sampling strategy in real time, effectively copes with complex operating environments, and significantly improves the safety and flexibility of sampling operations.

One or more embodiments of the present disclosure provide a lightweight and compact lunar regolith sampling system mountable on a rover, comprising: a three-degree-of-freedom manipulator based on open cylindrical thin-walled rods and a control unit based on three-dimensional force/torque information.

In some embodiments, the three-degree-of-freedom manipulator based on the open cylindrical thin-walled rods includes a translation mechanism, a side rotation mechanism, and a rear rotation mechanism, the translation mechanism, the side rotation mechanism, and the rear rotation mechanism provide a vertical motion, a left-right swing function, and a forward-backward swing function of a sampler, respectively; a sensor and the sampler are connected below the three-degree-of-freedom manipulator based on the open cylindrical thin-walled rods.

In some embodiments, the control unit based on three-dimensional force/torque information includes a data acquisition card and a mini computer NUC, wherein the data acquisition card acquires force feedback information fed back by a three-dimensional force/torque sensor and sends the force feedback information to the mini computer NUC via serial communication, and the mini computer NUC reads the three-dimensional force/torque information transmitted by the data acquisition card via the serial communication, reads an output shaft position of a servo motor via CAN bus communication, generates a real-time control instruction in combination with a compliance control algorithm, and sends the real-time control instruction to the servo motor via the CAN bus communication to implement a control closed loop.

In some embodiments, the translation mechanism includes a pair of storage boxes, a pair of open cylindrical thin-walled rods, two sets of concave-convex roller pairs, a bevel gear set, and a housing, uses the servo motor under position control as a driving device, wherein an output shaft of the servo motor is fixed to an internal rotating shaft of one of the convex rollers via a coupling, a body of the servo motor is fixed inside the housing, and a translational movement of the sampler is realized by driving contraction of the thin-walled rods.

In some embodiments, the pair of storage boxes are symmetrically fixed above an inside of the housing and are configured to limit and store the open cylindrical thin-walled rods.

In some embodiments, the open cylindrical thin-walled rods are spiral spring retractable flexible arms, are made of spring steel, are installed in the pair of storage boxes, respectively, each open cylindrical thin-walled rod extends downward through a small opening below the housing, and a surface of each open cylindrical thin-walled rod is provided with an equally spaced circular through-hole array formed by laser cutting.

In some embodiments, each of the two sets of the concave-convex roller pair has same arc surfaces as the open cylindrical thin-walled rods, wherein a central circumference of each convex roller is a toothed surface distributed with equally spaced small cylindrical teeth, a tooth pitch is consistent with a hole pitch of a surface of each open cylindrical thin-walled rod, a central circumference of each concave roller is a grooved surface, a groove surface depth is consistent with a small tooth height, the concave roller and the convex roller are symmetrically fixed on a support seat inside the housing via bearings, distributed on two sides of each open cylindrical thin-walled rod, the rod surface is pressed by adjusting a distance between the concave roller and the convex roller, and the small teeth on the central circumference of the convex roller engage with the holes on the rod surface.

In some embodiments, the bevel gear set is fixed outside the housing, the bevel gear set includes a pair of parallel bevel gears in a vertical direction, a pair of collinear bevel gears in a horizontal direction, and an optical axis, wherein the pair of collinear bevel gears in the horizontal direction are symmetrically connected in series on the optical axis and meshed with the pair of parallel bevel gears to realize rotational linkage of the bevel gear set, and the pair of parallel bevel gears are fixed to external axles of the two convex rollers via couplings to realize combined rotational movement of the two convex rollers.

In some embodiments, the housing includes a rotating shaft protruding outward from a side, and is fixed to the side rotation mechanism.

In some embodiments, two rotation mechanisms include a rotation mechanism providing a forward and backward swing function and a rotation mechanism providing a left-right swing function, each of the two rotation mechanisms include a servo motor and a motor housing.

In some embodiments, a side motor housing of the side rotation mechanism serves as a rotating blade, encapsulates a side servo motor, and an output shaft of the side servo motor is fixed to the rotating shaft on a side of the housing of the translation mechanism.

In some embodiments, an output shaft of a rear servo motor of the rear rotation mechanism is fixed to the side motor housing of the side rotation mechanism via a coupling, an axial direction of the rear servo motor is perpendicular to an axial direction of the side rotation mechanism, a body of the rear servo motor is fixed in a rear motor housing, and the rear motor housing simultaneously serves as an end of the manipulator and is mounted on a vehicle body of the rover.

In some embodiments, the sensor is the three-dimensional force/torque sensor, monitors a Z-axis force Fz, and X-axis and Y-axis torques Mx, My, and is installed at an end of a translation joint, wherein the Z axis is collinear with a motion direction of the translation joint, and the X-axis and the Y-axis are respectively parallel to rotating shafts of the side rotation mechanism and the rear rotation mechanism.

In some embodiments, the sampler is a mini drilling or digging sampler adapted to a shallow lunar soil sampling task.

In some embodiments, a control process of the compliance control algorithm includes:

S1: the mini computer NUC reading information of the three-dimensional force/torque sensor via the data acquisition card, and obtaining a used external force vector after low-pass filtering:

Fₑ=[Fz Mx My];

S2: selecting a damping-mass admittance control model without stiffness term as a motion equation, and performing compliance control in a joint space, wherein θᵣ is a reference position vector defined before a task, B and M are three-dimensional damping and mass coefficient matrices respectively, θ_{d} is a desired position vector of servo motors in a joint coordinate system, Δθ is a compliance deviation value, and s is a differential operator:

Fₑ=(M s²+Bs)(θᵣ−θ_{d})

E_{θ}=θᵣ−θ_{d}

E_{θ}=Fₑ/(M s²+Bs)

S3: running the above model in the mini computer NUC to obtain a real-time desired motor angle vector θ_{d}=[θz θx θy], wherein θz, θx, and θy serve as target angles, and are respectively transmitted to the servo motors in the translation mechanism, the side rotation mechanism, and the rear rotation mechanism via the CAN bus communication, thereby completing motion control to achieve an avoidance motion effect of complying with a resistance direction and realizing environmental adaptation.

### BRIEF DESCRIPTION OF THE DRAWINGS

FIG. 1 is a block diagram illustrating an exemplary sampling system according to some embodiments of the present disclosure.

FIG. 2 is a schematic diagram illustrating an exemplary translation mechanism according to some embodiments of the present disclosure.

FIG. 3 is a schematic diagram illustrating an exemplary concave-convex roller pair according to some embodiments of the present disclosure.

FIG. 4 is a schematic diagram illustrating an exemplary bevel gear set according to some embodiments of the present disclosure.

FIG. 5 is a schematic diagram illustrating an exemplary rotation mechanism according to some embodiments of the present disclosure.

FIG. 6 is a schematic diagram illustrating an exemplary control process according to some embodiments of the present disclosure.

### LIST OF REFERENCE NUMERALS IN THE DRAWINGS

1, translation mechanism; 2, side rotation mechanism; 3, rear rotation mechanism; 4, sensor; 5, sampler; 6, control unit; 11, storage box; 12, thin-walled rod; 121, rod surface hole; 13, concave-convex roller pair; 102, concave roller; 103, convex roller; 14, bevel gear set; 15, housing; 151, side rotating shaft; 16, translation servo motor; 17, side servo motor; 18, rear servo motor; 131, small tooth; 132, grooved surface; 133, bearing; 134, support seat; 141, parallel bevel gear; 142, collinear bevel gear; 143, optical axis; 21, side motor housing; 22, side servo motor output shaft; 31, rear servo motor output shaft; 32, rear motor housing.

### DETAILED DESCRIPTION

The present disclosure will be further elucidated below with reference to the accompanying drawings and the detailed embodiments. It should be understood that the following detailed embodiments are merely for illustrating the present disclosure and are not intended to limit the scope of the present disclosure.

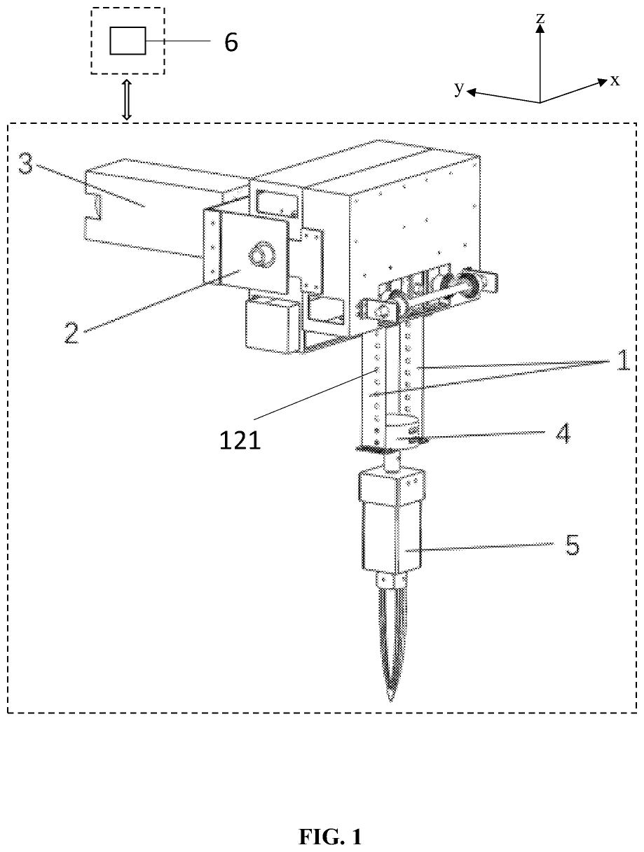

FIG. 1 is a schematic diagram illustrating an exemplary sampling system according to some embodiments of the present disclosure.

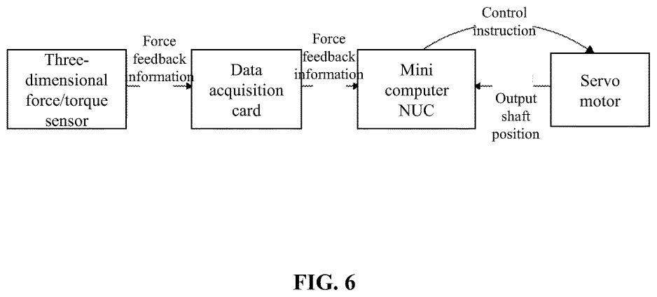

According to an aspect of the present disclosure, a lightweight and compact lunar regolith sampling system mountable on a rover is provided. The system includes a three-degree-of-freedom manipulator based on open cylindrical thin-walled rods (hereinafter referred to as thin-walled rods) and a control unit based on three-dimensional force/torque information. More descriptions regarding the control unit based on three-dimensional force/torque information may be found elsewhere in the present disclosure (e.g., FIG. 6 and related descriptions thereof).

In some embodiments, as shown in FIG. 1, the three-degree-of-freedom manipulator based on the thin-walled rods includes a translation mechanism 1, a side rotation mechanism 2, and a rear rotation mechanism 3, which are respectively configured to provide a vertical motion, a left-right swing function, and a forward-backward swing function of a sampler. A sensor 4 and a sampler 5 are connected below the manipulator.

The manipulator refers to a mechanical device of the sampling system configured to perform sampling operations. In some embodiments, the sampling system realizes, through the manipulator, flexible movement of the sampler in vertical, left-right, and forward-backward directions.

The phrase ‘based on open cylindrical thin-walled rods’ refers to the manipulator configured to realize flexible movement of the sampler in a plurality of directions by using the thin-walled rods as core motion actuators.

The translation mechanism 1 refers to a motion device configured to drive the sampler 5 to perform vertical motion (i.e., in a Z-axis direction).

The side rotation mechanism 2 refers to a mechanism configured to provide the sampler 5 with a forward-backward swing function (i.e., swinging about an X-axis).

The rear rotation mechanism 3 refers to a mechanism configured to provide the sampler 5 with a left-right swing function (i.e., swinging about an Y-axis).

The sensor 4 refers to a sensor configured to monitor force/torque of the sampling system in various directions.

The sampler 5 refers to an actuator configured to perform sampling operations.

More descriptions regarding the translation mechanism 1, the side rotation mechanism 2, the rear rotation mechanism 3, the sensor 4, and the sampler 5 may be found in the following related descriptions.

The lightweight and compact lunar regolith sampling system mountable on a rover designed in the present disclosure has a two-degree-of-freedom joint with a forward-backward swing degree of freedom and a left-right swing degree of freedom, and a linear translation joint. The sampling system has a compact design, a small volume, and a large and flexible motion space. The sensor and the sampler are directly connected below the manipulator, which facilitates real-time perception of operating force/torque information, thereby providing a basis for subsequent compliance control, and significantly improving the safety and adaptability of sampling operations.

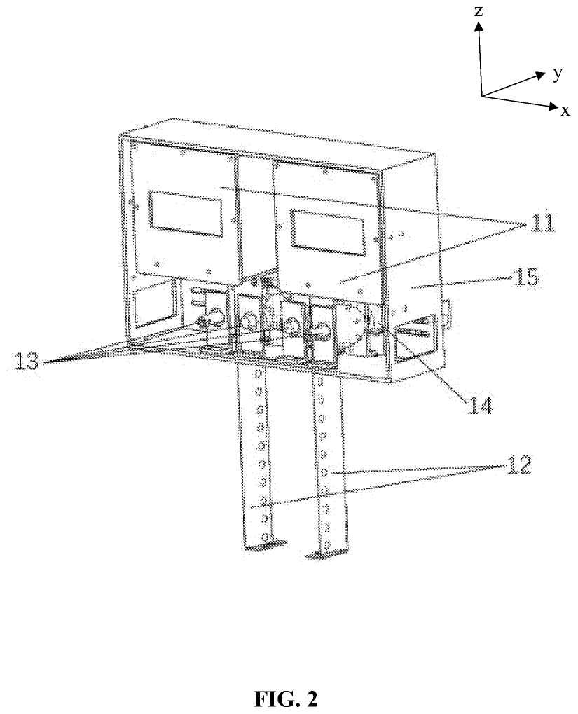

FIG. 2 is a schematic diagram illustrating an exemplary translation mechanism according to some embodiments of the present disclosure.

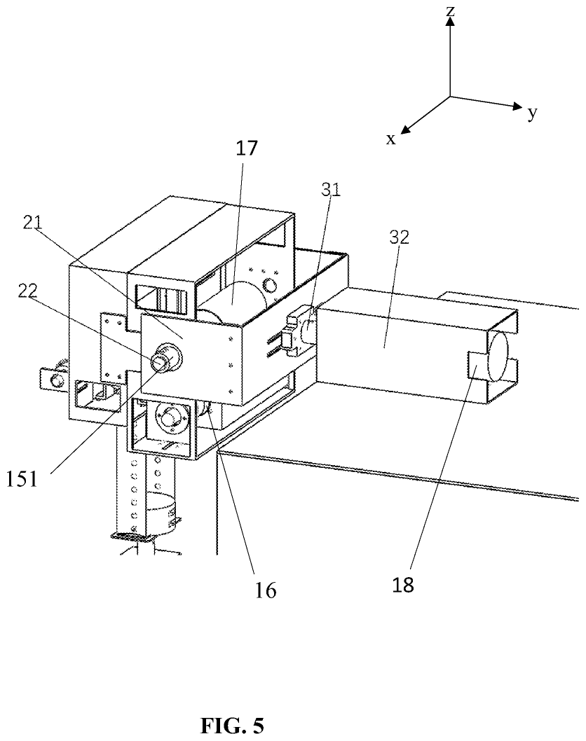

In some embodiments, as shown in FIG. 2, the translation mechanism 1 includes a pair of storage boxes 11, a pair of thin-walled rods 12, two sets of concave-convex roller pairs 13, a bevel gear set 14, and a housing 15, and uses a servo motor 16 under position control as a driving device (see FIG. 5), wherein an output shaft of the servo motor is fixed to an internal rotating shaft of one of the convex rollers via a coupling, a body of the servo motor is fixed inside the housing 15, and a translational movement of the sampler 5 is realized by driving contraction and extension of the thin-walled rods 12.

The servo motor 16 in the translation mechanism 1 (also referred to as a translation servo motor 16) refers to a motor configured to provide power for the translational movement of the thin-walled rods 12.

The position control refers to a control manner that takes precisely reaching a designated target position as a control objective, and realizes movement and stopping of an actuator (e.g., an output shaft of a servo motor) at a desired angle or displacement point through closed-loop regulation.

In some embodiments, the storage boxes 11, which are a pair, are symmetrically fixed above an inside of the housing 15, and are configured to limit and store the thin-walled rods 12.

In some embodiments, the storage boxes 11 may be made of lightweight high-strength materials (e.g., aluminum alloy or carbon fiber composite material). When the thin-walled rods 12 contract, the storage boxes 11 provide an internal winding space, allowing the thin-walled rods 12 to coil into a spiral shape from an extended state and be stored inside the boxes by relying on their inherent elasticity. Meanwhile, an opening end of the storage boxes 11 is provided with a guiding fillet, configured to guide the thin-walled rods 12 to automatically center during an extension process, thereby reducing friction and wear. The opening end refers to a port on the storage boxes 11 for the thin-walled rods 12 to extend or retract.

The thin-walled rods 12 are spiral spring retractable flexible arms, are made of spring steel, are installed in the pair of storage boxes, respectivelyes 11, extend downward through a small opening below the housing 15, and a surface of each thin-walled rod is provided with an equally spaced circular through-hole array formed by laser cutting.

The thin-walled rods 12 made of spring steel have characteristics of large axial stiffness and small contraction volume, which can ensure the straightness of the thin-walled rods 12 during an extension process. In some embodiments, the thin-walled rods 12 may also use other materials that have high elastic limits, good fatigue resistance, and coilable characteristics, such as beryllium bronze strips, titanium alloy thin strips, or carbon fiber reinforced resin matrix composite materials, or the like.

The small opening refers to a channel opening at a bottom of the housing 15 for the thin-walled rods 12 to extend or retract. In some embodiments, a shape of the small opening may be adapted to a cross-sectional shape of the thin-walled rods 12, such as a waist-shaped hole or a rectangular hole, or the like.

The through-hole array refers to a combination of through-holes configured to engage with small teeth 131 on the concave-convex roller pairs 13 to realize power transmission. In some embodiments, a shape of the through-holes is not limited to a circular shape, and other shapes such as an elliptical shape or a regular polygonal shape, or the like, may also be selected. In some embodiments, the through-hole array may also be obtained by manners such as stamping, electrical discharge machining, or precision mechanical drilling, or the like.

In some embodiments, each of the thin-walled rods 12 include a fixed end and a free end. The fixed end refers to an end of the thin-walled rod 12 that is fixedly connected inside the storage boxes 11 and whose position remains unchanged. The free end refers to an end of the thin-walled rod 12 that, relative to the fixed end, can extend or retract to connect to the sampler 5. In some embodiments, the fixed end of the thin-walled rod 12 is fixedly connected inside the storage boxes 11 via a connecting member (e.g., a rivet or a pressure plate), and the free end extends from an inner cavity of the storage boxes. In an initial storage state, the thin-walled rods 12 are coiled like a spiral spring or naturally curled inside the storage boxes 11. When driven to extend, the thin-walled rods 12 are gradually pulled out from the storage boxes 11 and expanded into cylindrical rod members having load-bearing capacity by relying on a tooth-hole engagement force of the concave-convex roller pairs 13. When the thin-walled rods 12 are driven to retract, the thin-walled rods 12 restore a curled form by relying on their inherent elasticity and are re-coiled into the storage boxes 11, thereby realizing compact storage.

The lightweight and compact lunar regolith sampling system mountable on a rover designed in the present disclosure uses the thin-walled rods 12 as a flexible translation mechanism, has advantages of a large extension stroke and high axial strength, is small in size and light in weight, which facilitates mounting on the rover to carry out sampling operations, and greatly reduces transportation costs.

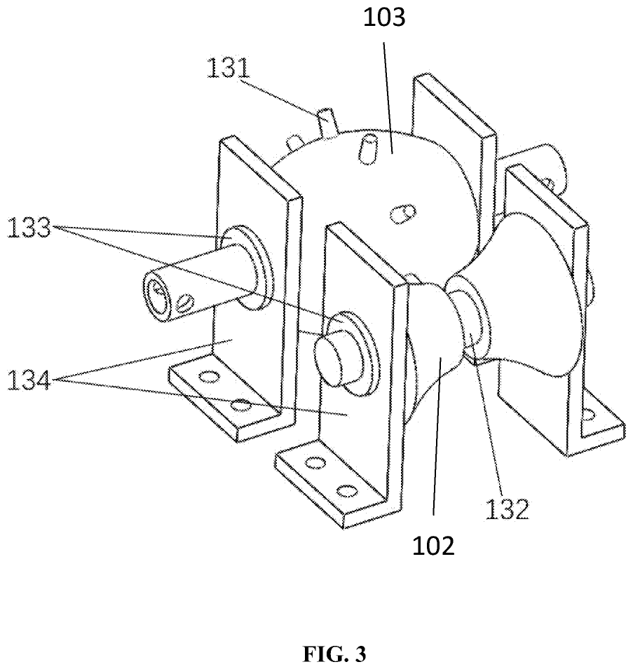

FIG. 3 is a schematic diagram illustrating a concave-convex roller pair according to some embodiments of the present disclosure.

The concave-convex roller pair 13 refers to a driving element configured to drive the thin-walled rod 12 to perform the translational movement. In some embodiments of the present disclosure, the concave-convex roller pair 13 includes a convex roller 103 and a concave roller 102. The small teeth 131 on the convex roller 103 engage with the holes 121 on the rod surface of the thin-walled rod 12, and cooperate with the concave roller 102 to press the rod surface, thereby transmitting motion. The convex roller 103 refers to a roller whose circumferential side surface protrudes outward among the concave-convex roller pair 13. The concave roller 102 refers to a roller whose circumferential side surface is inwardly recessed among the concave-convex roller pair 13. In some embodiments of the present disclosure, side surfaces of the convex roller 103 and the concave roller 102 are arc surfaces. In some embodiments of the present disclosure, as shown in FIG. 3, the concave-convex roller pair 13 has the same arc surface as the thin-walled rod 12, wherein a central circumference of the convex roller 103 is a toothed surface distributed with equally spaced small cylindrical teeth 131, and the tooth pitch is consistent with a hole pitch of the surface of the thin-walled rod 12. The central circumference of the concave roller 102 is a grooved surface 132, and a groove surface depth of the grooved surface 132 is consistent with a small tooth height. The concave roller and the convex roller are symmetrically fixed on the support seat 134 inside the housing via bearings 133, distributed on two sides of the thin-walled rod 12. The rod surface is pressed by adjusting a distance between the concave roller and the convex roller, and the small teeth on the central circumference of the convex roller 103 engage with the holes 121 on the rod surface.

The same arc surface refers to that the arc surfaces of the convex roller 103 and the concave roller 102 in the concave-convex roller pair 13 have the same radius of curvature as a cylindrical surface of the thin-walled rod 12, thereby allowing the two arc surfaces in the concave-convex roller pair 13 to closely fit with the cylindrical surface of the thin-walled rod 12, uniformly transmitting force during a driving process, avoiding local stress concentration, and effectively suppressing radial bending of the thin-walled rod 12, thereby ensuring linear motion accuracy of the thin-walled rod 12.

The central circumference refers to an annular band located at a middle position on the circumferential side surface of the convex roller 103 or the concave roller 102. The toothed surface refers to a surface provided with teeth (e.g., the small teeth 131).

The small teeth 131 refer to protrusions configured to engage with the through-holes on the surface of the thin-walled rod 12 to transmit motion. In some embodiments of the present disclosure, a shape of the small teeth 131 is not limited to a cylinder, and may also adopt conical teeth, trapezoidal teeth, or other protrusion structures capable of forming stable meshing engagement with the through-holes on the surface of the thin-walled rod 12. Meanwhile, the tooth pitch (i.e., the spacing between the adjacent small teeth), the small tooth height (i.e., the height of the small teeth 131), and a number of teeth of the small teeth 131 may be adaptively adjusted based on actual load and transmission ratio requirements.

The grooved surface 132 refers to an inwardly recessed mating surface on the central circumference of the concave roller 102. In some embodiments of the present disclosure, a cross-sectional shape of the grooved surface 132 (i.e., a cross-section in a circumferential direction perpendicular to the grooved surface) matches a profile of the small teeth 131, to allow the grooved surface 132 and the small teeth 131 to stably engage. In some embodiments of the present disclosure, the depth of the grooved surface 132 may also be inconsistent with the small tooth height, as long as the grooved surface and the small teeth can form stable engagement in an engaged state.

The support seat 134 refers to a supporting member configured to mount the concave-convex roller pair 13. In some embodiments of the present disclosure, the support seat 134 may be mounted on the inner wall of the housing 15. In some embodiments of the present disclosure, the support seat 134 is an L-shaped member, wherein one end of the L-shaped member is connected to the inner wall of the housing 15, and the other end is connected to the concave-convex roller pair 13 via bearings. In some embodiments of the present disclosure, axles at both ends of the convex roller 103 and the concave roller 102 are respectively interference fitted with inner rings of their respective bearings 133. Outer rings of the respective bearings 133 are embedded and fixed in corresponding mounting holes of the support seat 134, thereby realizing rotational support and axial positioning for the concave-convex roller pair 13. In some embodiments of the present disclosure, one end of the L-shaped member may be fixedly connected (e.g., screw connection or welding) to an inner wall of the housing 15 or movably connected thereto. The movable connection may be implemented by means of (e.g., an eccentric bushing or a slider).

In some embodiments of the present disclosure, as shown in FIG. 2, a concave-convex roller pair 13 is arranged on two opposing side surfaces of a thin-walled rod 12. The convex roller 103 and the concave roller 102 are oppositely arranged to clamp and drive the thin-walled rod 12 to perform the translational movement from both sides.

In some embodiments of the present disclosure, the bearings 133 of at least one roller in the concave-convex roller pair 13 are mounted on the movable support seat 134. By pushing the support seat 134 (e.g., by tightening an adjustment screw) to translate along a guide groove (e.g., a waist-shaped hole) within the housing 15, a radial distance between the convex roller 103 and the concave roller 102 is changed, thereby pressing the rod surface of the thin-walled rod 12.

The holes 121 on the rod surface refer to through-holes on the thin-walled rod 12, forming the aforementioned through-hole array. In some embodiments of the present disclosure, the small teeth 131 on the central circumference of the convex roller 103 engage with the holes 121 on the rod surface of the thin-walled rod 12. When the convex roller 103 is driven to rotate, the equally spaced small teeth 131 distributed on the central circumference of the convex roller 103 sequentially embed in the corresponding through-holes on the surface of the thin-walled rod 12. Through contact pressure between side surfaces of the small teeth and hole walls of the through-holes, the circumferential driving force is transmitted, thereby converting the rotational movement of the convex roller 103 into the translational movement of the thin-walled rod 12, to achieve non-slipping tooth-hole meshing transmission. The circumferential driving force refers to a force that pushes the thin-walled rod 12 to perform linear movement along a circumferential tangential direction of the convex roller 103.

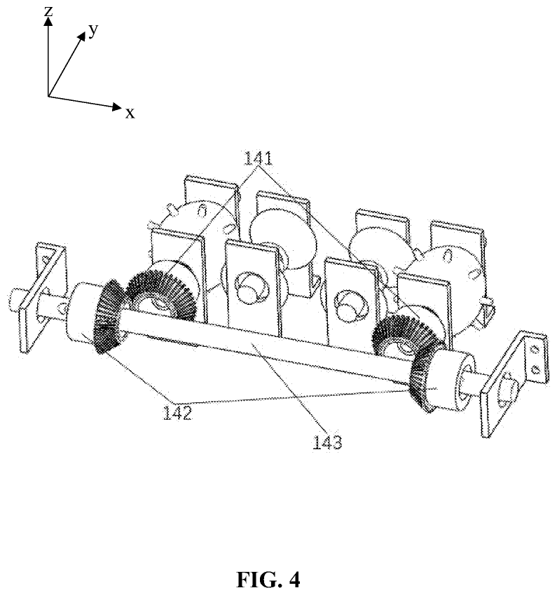

FIG. 4 is a schematic diagram illustrating a bevel gear set according to some embodiments of the present disclosure.

The bevel gear set 14 refers to a transmission mechanism configured to synchronously transmit a single power source to the two convex rollers 103 to realize combined rotation. The bevel gear refers to a type of gear with a conical toothed surface, configured to transmit rotational movement and torque between two intersecting shafts. In some embodiments of the present disclosure, as shown in FIG. 4, the bevel gear set 14 is fixed outside the housing 15, and includes a pair of parallel bevel gears 141 in a vertical direction (i.e., a y-direction), a pair of collinear bevel gears 142 in a horizontal direction (i.e., an x-direction), and an optical axis 143. The pair of collinear bevel gears 142 in the horizontal direction are symmetrically connected in series on the optical axis 143 and meshed with the pair of parallel bevel gears 141, to realize rotational linkage of the bevel gear set 14. The parallel bevel gears 141 are fixed to external axles of the two convex rollers via couplings (not shown), to realize combined rotational movement of the two convex rollers.

In some embodiments of the present disclosure, the bevel gear set 14 may be fixed outside the housing 15 by means of bearing connections (e.g., rolling bearings, sliding bearings, or the like) or flange plates, or the like. For example, the bevel gear set 14 may be fixed to an outer side wall surface of the housing 15 by means of rolling bearings mounted at both ends of the optical axis 143 and corresponding bearing seats.

The parallel bevel gear 141 refers to a pair of bevel gears whose axes are parallel to each other. The collinear bevel gear 142 refers to a pair of bevel gears whose axes are collinear.

The optical axis 143 refers to a long straight shaft configured to connect in series and fix the collinear bevel gears 142 to enable them to synchronously rotate. In some embodiments of the present disclosure, the collinear bevel gears 142 may be symmetrically fixed in series on the optical axis 143 by means of key connections or interference fits, or the like, and may be axially positioned by shaft shoulders or retaining rings, or the like.

The coupling (also referred to as a coupler) refers to a device configured to connect two shafts or a shaft and a rotating member, and to transmit movement and torque. The coupling includes, but is not limited to, flange couplings, sleeve couplings, and split-muff couplings. In some embodiments of the present disclosure, the coupling includes two half couplings and locking elements configured to connect the two half couplings (e.g., set screws, parallel keys, or shrink disks, or the like). A half coupling includes mounting holes configured to insert a shaft or a rotating member.

In some embodiments of the present disclosure, a hub end extended shaft of the parallel bevel gear 141 and the external axle of the convex roller are respectively inserted from both ends into mounting holes of two half couplings of the same coupling, and locked by locking elements, thereby realizing coaxial rigid connection between the bevel gear and the convex roller 103 to transmit the rotational movement and torque.

In some embodiments of the present disclosure, as shown in FIG. 4, in the bevel gear set 14, one of the convex rollers 103 (e.g., the left side convex roller 103) is connected to the translation servo motor 16. The translation servo motor 16 drives the left side convex roller 103 to rotate, which in turn drives the left side parallel bevel gear 141 connected to the left side convex roller 103 via a coupling to rotate. The left side parallel bevel gear 141 and the left side collinear bevel gear 142 in the horizontal direction mesh with each other, thereby transmitting power to the optical axis 143. The two collinear bevel gears 142 symmetrically connected in series on the optical axis 143 synchronously rotate, and the right side collinear bevel gear 142 then meshes with the right side parallel bevel gear 141, thereby driving the external axle of the right side convex roller 103 to rotate in reverse. Through the aforementioned gear meshing chain, the bevel gear set 14 synchronously and oppositely transmits the rotational movement of a single motor to the two convex rollers 103, causing the two convex rollers 103 to jointly rotate at the same speed and in reverse, to realize stable driving of the thin-walled rod 12. The left side and right side as described in the present disclosure are distinctions made with respect to the axial direction of the optical axis 143, indicating the symmetrical arrangement positions of the two collinear bevel gears 142 and the two convex rollers 103 along the axial direction of the optical axis.

The housing 15 refers to a casing structure configured to house and support internal components such as the storage box 11, the concave-convex roller pair 13, and the translation servo motor 16, or the like. In some embodiments of the present disclosure, as shown in FIG. 5, the housing 15 includes a rotating shaft 151 protruding outward from a side, and is fixed to the side rotation mechanism 2.

The rotating shaft 151 refers to a support shaft configured to realize the forward and backward swing of the sampler 5. In some embodiments of the present disclosure, a number of the rotating shafts 151 are 2. The two rotating shafts 151 are fixedly connected (e.g., screw connection or welding, or the like) on outer side walls of the housing 15 on two sides along the x-direction. In some embodiments of the present disclosure, the rotating shaft 151 includes accommodation holes configured to accommodate the output shaft 22 of the side servo motor 17. In some embodiments of the present disclosure, the housing 15 is fixedly connected to the output shaft 22 of the side servo motor 17 via the rotating shaft 151, and is further relatively fixed to the side rotation mechanism 2.

In some embodiments of the present disclosure, by utilizing the spiral spring retractable characteristic of the thin-walled rod 12, combined with tooth-hole meshing transmission of the concave-convex roller pair 13 and synchronous reverse drive of the bevel gear set 14, a lightweight motion solution with large stroke telescopic movement and small volume storage is realized. Meanwhile, the pressing by the concave-convex roller pair 13 and precise tooth-hole engagement ensure transmission stability and load capacity, thereby effectively improving the integration degree and reliability of the sampling system in deep space exploration missions.

FIG. 5 is a schematic diagram illustrating an exemplary rotation mechanism according to some embodiments of the present disclosure.

In some embodiments, as shown in FIG. 5, the two rotation mechanisms 2 and 3 (i.e., the side rotation mechanism 2 and the rear rotation mechanism 3) include the side rotation mechanism 2 configured to provide the forward and backward swing function (swinging about the X-axis) and the rear rotation mechanism 3 configured to provide the left-right swing function (swinging about the Y-axis), and each of the rotation mechanisms includes a servo motor and a motor housing.

In some embodiments, the side motor housing 21 of the side rotation mechanism 2 serves as a rotating blade, encapsulates the side servo motor 17, and the output shaft 22 of the side servo motor 17 is fixed to the rotating shaft 151 on a side of the housing 15 of the translation mechanism.

In some embodiments, the side rotation mechanism 2 includes the side motor housing 21 and the side servo motor 17. The side motor housing 21 refers to a box body device configured to receive the side servo motor 17. The side servo motor 17 refers to a motor configured to provide power for forward and backward swinging of the sampler 5.

The rotating blade refers to the side motor housing 21 serving as a swing arm, which swings left and right (about the Y-axis) under the drive of the output shaft 31 of the rear servo motor 18.

In some embodiments, the output shaft 22 of the side servo motor 17 may be inserted into a receiving hole of the rotating shaft 151 and fixed (e.g., by interference fit, radial screw locking, or circumferential key fixing, or the like), thereby allowing reliable transmission of the torque.

In some embodiments, the output shaft 31 of the rear servo motor 18 of the rear rotation mechanism 3 is fixed to the side motor housing 21 of the side rotation mechanism 2 via a coupling, and the axial direction of the rear rotation mechanism 3 is perpendicular to the axial direction of the side rotation mechanism 2. The body of the rear servo motor 18 is fixed (e.g., by screw connection or welding, or the like) in the rear motor housing 32, and the rear motor housing 32 may simultaneously serve as the end of the manipulator and be mounted on the vehicle body of the rover.

In some embodiments, the rear rotation mechanism 3 includes the rear motor housing 32 and the rear servo motor 18. The rear motor housing 32 refers to a box body device configured to receive the rear servo motor 18. The rear servo motor 18 refers to a motor configured to provide power for left-right swinging of the sampler 5.

The axial direction of the rear rotation mechanism 3 refers to the axial direction along the output shaft 31 of the rear servo motor 18. The axial direction of the side rotation mechanism 2 refers to the axial direction along the output shaft 22 of the side servo motor 17.

The body of the rear servo motor 18 refers to the main body portion of the motor other than the output shaft 31.

In some embodiments, an outer wall of the rear motor housing 32 may be fixedly mounted (e.g., integrally formed or welded, or the like) with a plurality of mounting lugs (not shown in the figures). Each of the mounting lugs is provided with a through-hole, and is fixedly connected to corresponding brackets on the vehicle body of the rover via bolts, thereby allowing secure mounting of the entire sampling system on the rover.

In some embodiments, when the rear servo motor 18 is driven, the output shaft 31 of the rear servo motor 18 drives the side motor housing 21 to rotate about the Y-axis (i.e., left-right swinging), which further drives the entire side rotation mechanism 2 and the translation mechanism 1 to swing left and right about the Y-axis. When the side servo motor 17 is driven, the output shaft 22 of the side servo motor 17 drives the housing 15 to rotate about the X-axis (i.e., forward and backward swinging), which further drives the translation mechanism 1 together with the sampler 5 to swing forward and backward about the X-axis. The two rotation mechanisms operate collaboratively to realize attitude adjustment of the sampler in the left-right and forward-backward directions.

In some embodiments of the present disclosure, the side rotation mechanism 2 and the rear rotation mechanism 3 adopt an axially perpendicular arrangement, and the rear motor housing 32 also serves as the end of the manipulator and is mounted on the vehicle body of the rover, thereby realizing spatial decoupling of the two degrees of rotational freedom and simple and reliable motion control. The side motor housing 21 serves as a swing arm to directly drive the translation mechanism 1, having a compact structure and a short transmission chain. The rear motor housing 32 integrates a mounting function, eliminating the need for an additional bracket, which further reduces the system weight and saves the rover mounting space. Overall, a manipulator joint module that is small in volume, light in weight, has a large swing range, and is flexible in motion is constructed, significantly improving the attitude adjustment capability and operational adaptability of the sampling system in complex lunar surface environments.

In some embodiments, as shown in FIG. 1, the sensor 4 is the three-dimensional force/torque sensor, monitors a Z-axis force Fz, and X-axis and Y-axis torques Mx, My, and is installed at an end of the translation joint (also referred to as the translation mechanism 1). The Z-axis is collinear with the motion direction of the translation joint, and the X-axis and the Y-axis are respectively parallel to the rotating shafts of the side rotation mechanism 2 and the rear rotation mechanism 3.

The three-dimensional force/torque sensor may measure force and/or torque in three directions (the Z-axis, the X-axis, and the Y-axis). The Z-axis, the X-axis, and the Y-axis refer to three preset and measurable mutually perpendicular directions on the three-dimensional force/torque sensor. In some embodiments, the three-dimensional force/torque sensor may include, but is not limited to, a strain gauge type, a piezoelectric type, or a capacitive type, or the like. Its specific range, accuracy, and dimensions may be adaptively configured based on the rover load and the sampling task requirements.

The end of the translation joint refers to the free end of the thin-walled rod 12 in the translation mechanism 1. In some embodiments, as shown in FIG. 1, the sensor 4 may be fixedly mounted (e.g., by screw connection or snap connection, or the like) on the free end of the thin-walled rod 12, to move synchronously with the sampler 5.

The motion direction of the translation joint refers to the direction of vertical motion of the translation mechanism 1.

In some embodiments, it may be achieved by mounting arrangements that the Z-axis on the sensor 4 is collinear with the motion direction of the translation joint (i.e., the z-axis), the X-axis is collinear with the output shaft 22 of the side servo motor 17 in the side rotation mechanism 2 (i.e., the x-axis), and the Y-axis is collinear with the output shaft 31 of the rear servo motor 18 in the rear rotation mechanism 3 (i.e., the y-axis).

In some embodiments, the sensor 4 is the three-dimensional force/torque sensor. When the sampler 5 contacts the lunar regolith, the force generated in the vertical direction (i.e., the z-axis direction) is directly transmitted to the sensor 4, thereby determining the Z-axis force Fz. Meanwhile, the torsional torques experienced by the sampler about the X-axis and the Y-axis are determined by differential output calculation of an internal strain structure or piezoelectric elements of the sensor 4 to obtain Mx and My.

In some embodiments of the present disclosure, by installing the three-dimensional force/torque sensor at the end of the translation joint, real-time precise acquisition of multi-dimensional force signals during the sampling process may be realized, thereby achieving real-time monitoring of the system operational state below the lunar regolith. Defining the Z-axis force to be collinear with the translational motion direction and the X/Y-axis torques to be respectively parallel to the rotating shafts of the side rotation mechanism and the rear rotation mechanism, thereby realizing a one-to-one correspondence between the motion directions of the respective mechanisms and the monitored force directions. This correspondence allows the compliance control algorithm to directly perform decoupled control in the joint space without complex coordinate transformations, simplifying the control model, and improving the real-time performance and control accuracy of force feedback, thereby more reliably realizing obstacle avoidance and environmental adaptation.

In some embodiments, the sampler 5 is a mini drilling or digging sampler adapted to a shallow lunar soil sampling task.

In some embodiments, the sampler 5 may be selected as a helical drilling sampler or a bucket-type digging sampler based on the specific lunar soil sampling task requirements. A drilling sampler obtains shallow lunar regolith samples (e.g., 0 to 50 cm in depth) through rotary drilling, while a digging sampler is suitable for digging loose surface lunar regolith. The above-mentioned sampler 5 is rigidly connected to the sensor 4. The three-dimensional force/torque information generated by the sampler 5 during the operational process is acquired in real-time by the sensor 4 and fed back to the control unit, for closed-loop adjustment of the manipulator attitude, to avoid damaging the sampler due to excessive resistance.

FIG. 6 is a schematic diagram illustrating an exemplary control process according to some embodiments of the present disclosure.

In some embodiments, as shown in FIG. 6, the control unit based on three-dimensional force/torque information includes the data acquisition card and the NUC. The data acquisition card acquires force feedback information fed back by the three-dimensional force/torque sensor and sends the force feedback information to the NUC via serial communication. The NUC reads the three-dimensional force/torque information transmitted by the data acquisition card via the serial communication, reads an output shaft position of the servo motor via CAN bus communication, generates a real-time control instruction in combination with the compliance control algorithm, and sends the real-time control instruction to the servo motor via the CAN bus communication to implement a control closed loop.

The three-dimensional force/torque information refers to force feedback information acquired in real time by a three-dimensional force/torque sensor, including a Z-axis force Fz, an X-axis torque Mx, and a Y-axis torque My.

‘Based on three-dimensional force/torque information’ refers to the control unit 6 achieving real-time closed-loop control of the manipulator by using the three-dimensional force/torque information acquired by the three-dimensional force/torque sensor as a core feedback quantity.

The control unit 6 refers to a system module configured to generate and send instructions to drive the motion of the manipulator. In some embodiments, the control unit may, based on the three-dimensional force/torque information and the compliance control algorithm, generate and send instructions in real time to drive the servo motors of respective mechanisms of the manipulator to implement closed-loop motion control. In some embodiments, the control unit 6 may be mounted on the vehicle body of the rover or integrated near the rear motor housing 32 of the manipulator. In some embodiments, the control unit 6 may be communicatively connected to respective servo motors in the translation mechanism 1, the side rotation mechanism 2, and the rear rotation mechanism 3.

The data acquisition card refers to a hardware module configured for data acquisition. The mini computer NUC refers to a processing module configured for data analysis, processing, and instruction generation and distribution.

The serial communication refers to a communication interface technology that transmits data bit by bit in a serial manner, and is widely applied for low-speed, simple, and reliable data exchange between devices.

The Controller Area Network (CAN) bus communication is a serial communication technology based on a Controller Area Network (CAN) protocol, configured to transmit control instructions and position data between the NUC and the servo motors.

The output shaft position of the servo motor refers to a rotation angle of the output shaft of the servo motor. In some embodiments, an encoder may be provided inside each of the servo motors. The encoder may measure a rotation angle of an output shaft of the servo motor, and the NUC may read the rotation angle of the output shaft of the servo motor from the encoder via CAN bus communication. The encoder refers to a rotary sensor that can convert a rotational displacement into a series of digital pulse signals, and the pulse signals are used to control an angular displacement.

In some embodiments, since the data volume of the three-dimensional force/torque information is small and the transmission rate requirement is not high, and a serial communication protocol is simple and easy to implement, and both are usually near-field point-to-point connections with low cost and high reliability, therefore, serial communication may be used between the NUC and the data acquisition card. Servo motor control of various mechanisms requires high real-time performance, multi-node coordination, and anti-interference capability. CAN bus supports multi-master multi-slave and differential signal transmission, which can ensure accurate and rapid sending and receiving of synchronous control instructions and position feedback for a plurality of servo motors, thereby adapting to stable communication requirements in complex lunar surface environments. Therefore, CAN bus communication may be adopted between the NUC and the respective servo motors.

The compliance control algorithm refers to an algorithm configured to adjust joint motion of the manipulator in real time to comply with external environmental resistance.

In some embodiments of the present disclosure, the control unit 6 generates closed-loop instructions by acquiring three-dimensional force/torque information in real time and combining it with the compliance control algorithm, allowing the manipulator to perceive an operating environment below the lunar regolith and actively adjust its posture to comply with a resistance direction, thereby effectively avoiding obstacles and preventing structural damage, and significantly improving the safety and environmental adaptability of a sampling process.

In some embodiments, the control process of the compliance control algorithm includes:

S1: The NUC reading the three-dimensional force/torque sensor information via the data acquisition card, and obtaining the external force vector Fₑ obtained after the low-pass filtering (e.g., first-order low-pass filtering or sliding average filtering, or the like):

Fₑ=[Fz Mx My];

S2: selecting a damping-mass admittance control model without stiffness term as a motion equation, and performing compliance control in a joint space, wherein θᵣ is a reference position vector defined before a task, B and M are three-dimensional damping and mass coefficient matrices, respectively, θ_{d} is a desired position vector of the servo motor in a joint coordinate system, Δθ is a compliance deviation value, and s is a differential operator:

Fₑ=(M s²+Bs)(θᵣ−θ_{d})

E_{θ}=θᵣ−θ_{d}

E_{θ}=Fₑ/(M s²+Bs)

The admittance control model refers to a force control strategy configured to implement compliance interaction of the manipulator. The admittance control model takes an external force as an input, determines a desired position or velocity correction amount through a preset dynamic model (e.g., a mass-damping-spring (stiffness-related) system), thereby causing the manipulator to generate a compliant response to the external force. The damping-mass admittance control model without stiffness term refers to a simplified admittance control strategy, which only includes a damping matrix and a mass matrix, but does not include a stiffness matrix. Under this model, a motion response of the manipulator only depends on an external force and its own inertia and damping characteristics, and there is no elastic restoring force. When subjected to the external force, the manipulator will generate a velocity change and continuously move in compliance with a direction of the external force, instead of attempting to return to a fixed position. This design facilitates the sampling system performing compliance avoidance of an obstacle external force through posture adjustment, preventing damage to mechanisms, and improving the environmental adaptation capability of the sampling system.

The joint space (also referred to as the joint coordinate system) refers to a coordinate system space constituted by variables of the respective joints of the manipulator (e.g., a displacement of the translation mechanism 1, and rotation angles of rotation mechanisms (e.g., the side rotation mechanism 2 and the rear rotation mechanism 3)) as coordinate axes.

The reference position vector θᵣ refers to a position vector preset before a sampling task begins, configured to describe desired positions of the servo motors of respective joints (e.g., the translation mechanism 1, the side rotation mechanism 2, and the rear rotation mechanism 3). In some embodiments, the reference position vector is a vector of the servo motor in the joint coordinate system. In some embodiments, the reference position vector may be set by the control unit 6 based on environmental conditions (e.g., terrain data). The terrain data refers to data reflecting terrain conditions around the rover, and may be acquired by an imaging device on the rover.

The desired position vector θ_{d} refers to an instruction position vector in the joint coordinate system after a sampling task begins, configured for the respective servo motors as a real-time control target. In some embodiments, the desired position vector may be determined through the aforementioned admittance control model.

The compliance deviation value Δθ refers to a displacement correction amount for correcting the reference position vector in the joint coordinate system. In some embodiments, the compliance deviation value may be determined based on real-time detected three-dimensional force/torque information and through the admittance control model.

The differential operator refers to a symbol configured to represent differentiation/differential operation on variables, which can simplify the differential operation into a symbolic form for algebraic processing.

S3: running the model in the NUC to obtain a real-time desired motor angle vector (i.e., the desired position vector) θ_{d}=[θz θx θy], wherein θz, θx, and θy serve as target angles, and are respectively transmitted to the servo motors in the translation mechanism 1, the side rotation mechanism 2, and the rear rotation mechanism 3 via CAN bus communication, to complete motion control, thereby achieving an avoidance motion effect of complying with a resistance direction, and realizing environmental adaptation.

θz is the target angle of the translation mechanism 1; θx is the target angle of the side rotation mechanism 2; and θy is the target angle of the rear rotation mechanism 3. In some embodiments, the translation mechanism 1, the side rotation mechanism 2, and the rear rotation mechanism 3 respectively rotate by corresponding angles based on θz, θx, and θy to cause the manipulator to rotate to a target position. The target position refers to a spatial pose that the manipulator is desired to reach to comply with an external force and avoid rigid impact.

In some embodiments of the present disclosure, the control unit 6 implements one-to-one corresponding closed-loop control of force and motion direction in the joint space through the damping-mass admittance control model without stiffness term, allowing the manipulator to actively adjust its posture to comply with an external force direction based on real-time detected three-dimensional force/torque information, thereby effectively avoiding obstacles below the lunar regolith and preventing damage to a sampling mechanism due to rigid impact, and significantly improving environmental adaptability and safety of a sampling operation; meanwhile, the algorithm model is simple and has high computational efficiency, and is easy to run in real time on the NUC, thereby ensuring reliability and response speed of a control system.

It should be noted that the above content merely describes the technical idea of the present disclosure, and should not be construed as limiting the protection scope of the present disclosure. For a person skilled in the art, several improvements and modifications may be made without departing from the principles of the present disclosure, and these improvements and modifications shall fall within the protection scope of the claims of the present disclosure.

What is claimed is:

1. A lightweight and compact lunar regolith sampling system mountable on a rover, comprising: a three-degree-of-freedom manipulator based on open cylindrical thin-walled rods and a control unit based on three-dimensional force/torque information;
the three-degree-of-freedom manipulator based on the open cylindrical thin-walled rods includes a translation mechanism, a side rotation mechanism, and a rear rotation mechanism, the translation mechanism, the side rotation mechanism, and the rear rotation mechanism provide a vertical motion, a left-right swing function, and a forward-backward swing function of a sampler, respectively, wherein a sensor and the sampler are connected below the three-degree-of-freedom manipulator based on the open cylindrical thin-walled rods;
the control unit based on three-dimensional force/torque information includes a data acquisition card and a mini computer NUC, wherein the data acquisition card acquires force feedback information fed back by a three-dimensional force/torque sensor and sends the force feedback information to the mini computer NUC via serial communication, and the mini computer NUC reads the three-dimensional force/torque information transmitted by the data acquisition card via the serial communication, reads an output shaft position of a servo motor via CAN bus communication, generates a real-time control instruction in combination with a compliance control algorithm, and sends the real-time control instruction to the servo motor via the CAN bus communication to implement a control closed loop;
the translation mechanism includes a pair of storage boxes, a pair of open cylindrical thin-walled rods, two sets of concave-convex roller pairs, a bevel gear set, and a housing, uses the servo motor under position control as a driving device, wherein an output shaft of the servo motor is fixed to an internal rotating shaft of one of the convex rollers via a coupling, a body of the servo motor is fixed inside the housing, and a translational movement of the sampler is realized by driving contraction of the thin-walled rods;
the pair of storage boxes are symmetrically fixed above an inside of the housing and are configured to limit and store the open cylindrical thin-walled rods;
the open cylindrical thin-walled rods are spiral spring retractable flexible arms, are made of spring steel, are installed in the pair of storage boxes, respectively, each open cylindrical thin-walled rod extends downward through a small opening below the housing, and a surface of each open cylindrical thin-walled rod is provided with an equally spaced circular through-hole array formed by laser cutting;
each of the two sets of the concave-convex roller pairs has a same arc surface as the open cylindrical thin-walled rods, wherein a central circumference of each convex roller is a toothed surface distributed with equally spaced small cylindrical teeth, a tooth pitch is consistent with a hole pitch of a surface of each open cylindrical thin-walled rod, a central circumference of the concave roller is a grooved surface, a groove surface depth is consistent with a small tooth height, the concave roller and convex roller are symmetrically fixed on a support seat inside the housing via bearings, distributed on two sides of open cylindrical thin-walled rods, the rod surface is pressed by adjusting a distance between the concave roller and the convex roller, and the small teeth on the central circumference of the convex roller engage with the holes on the rod surface;
the bevel gear set is fixed outside the housing, the bevel gear set includes a pair of parallel bevel gears in a vertical direction, a pair of collinear bevel gears in a horizontal direction, and an optical axis, wherein the pair of collinear bevel gears in the horizontal direction are symmetrically connected in series on the optical axis and meshed with the pair of parallel bevel gears to realize rotational linkage of the bevel gear set, and the pair of parallel bevel gears are fixed to external axles of the two convex rollers via couplings to realize combined rotational movement of the two convex rollers; and
the housing includes a rotating shaft protruding outward from a side, and is fixed to the side rotation mechanism.

2. The lightweight and compact lunar regolith sampling system mountable on the rover according to claim 1, wherein two rotation mechanisms include a rotation mechanism providing a forward and backward swing function and a rotation mechanism providing a left-right swing function, each of the two rotation mechanisms include a servo motor and a motor housing;
a side motor housing of the side rotation mechanism serves as a rotating blade, encapsulates a side servo motor, and an output shaft of the side servo motor is fixed to the rotating shaft on a side of the housing of the translation mechanism;
an output shaft of a rear servo motor of the rear rotation mechanism is fixed to the side motor housing of the side rotation mechanism via a coupling, an axial direction of the rear servo motor is perpendicular to an axial direction of the side rotation mechanism, a body of the rear servo motor is fixed in a rear motor housing, and the rear motor housing simultaneously serves as an end of the manipulator and is mounted on a vehicle body of the rover.

3. The lightweight and compact lunar regolith sampling system mountable on the rover according to claim 1, wherein the sensor is the three-dimensional force/torque sensor, monitors a Z-axis force Fz, and X-axis and Y-axis torques Mx, My, and is installed at an end of a translation joint, wherein the Z axis is collinear with a motion direction of the translation joint, and the X-axis and the Y-axis are respectively parallel to rotating shafts of the side rotation mechanism and the rear rotation mechanism.

4. The lightweight and compact lunar regolith sampling system mountable on the rover according to claim 1, wherein the sampler is a mini drilling or digging sampler adapted to a shallow lunar soil sampling task.

5. The lightweight and compact lunar regolith sampling system mountable on the rover according to claim 1, wherein a control process of the compliance control algorithm includes:
S1: the mini computer NUC reading information of the three-dimensional force/torque sensor via the data acquisition card, and obtaining a used external force vector after low-pass filtering:
Fₑ=[Fz Mx My];
S2: selecting a damping-mass admittance control model without stiffness term as a motion equation, and performing compliance control in a joint space, wherein θᵣ is a reference position vector defined before a task, B and M are three-dimensional damping and mass coefficient matrices respectively, θ_{d} is a desired position vector of servo motors in a joint coordinate system, Δθ is a compliance deviation value, and s is a differential operator:
Fₑ=(M s²+Bs)(θᵣ−θ_{d})
E_{θ}=θᵣ−θ_{d}
E_{θ}=Fₑ/(M s²+Bs)
S3: running the above model in the mini computer NUC to obtain a real-time desired motor angle vector θ_{d}=[θz θx θy], wherein θz, θx, and θy serve as target angles, and are respectively transmitted to the servo motors in the translation mechanism, the side rotation mechanism, and the rear rotation mechanism via the CAN bus communication, thereby completing motion control to achieve an avoidance motion effect of complying with a resistance direction and realizing environmental adaptation.
